(19) United States
(12) Patent Application Publication
(10) Pub. No.: US 2012/0037526 A1
(43) Pub. Date: Feb. 16, 2012

(54) STORAGE AND DISPENSING SYSTEM FOR REUSABLE SYRINGE

(75) Inventors: Anthony Mulone, Marston Mills, MA (US); Kathleen Tremblay, Westfield, MA (US); Warren W. Howland-Copp, Chicopee, MA (US); Jeffrey T. Delnickas, Kingston, MA (US)

(73) Assignee: TYCO HEALTHCARE GROUP LP, Mansfield, MA (US)

(21) Appl. No.: 13/208,033

(22) Filed: Aug. 11, 2011

Related U.S. Application Data
(60) Provisional application No. 61/373,120, filed on Aug. 12, 2010.

(51) Int. Cl. A61B 19/02; A61J 1/00

(52) U.S. Cl. 206/366

(57) ABSTRACT

A storage and dispensing container includes a base having an open end and a receptacle. A first divider is secured in the receptacle. A second divider is pivotally secured to the first divider, and defines a first compartment with the first divider. The first compartment has a size that is selectively adjustable by pivoting the second divider relative to the first divider.

### CROSS REFERENCE TO RELATED APPLICATION

This application claims priority to the U.S. Patent Application 611373,120, filed Aug. 12, 2010, entitled, “Storage and Dispensing System for Reusable Syringe”, which is incorporated by reference in its entirety.

### BACKGROUND

The present invention generally relates to systems for storing and dispensing medical cartridges and disposable needles for use with reusable syringes, and more particularly to a container for storing and dispensing cartridges and needles, and for further disposing of the used needle assemblies.

Many injectable drugs are administered using a reusable syringe and disposable needle assemblies. Frequently, injectable drugs such as local anesthetics are provided in disposable cartridges. Usually, the disposable needles are provided in a first container and the drug cartridges are provided in a separate second container. A third container is provided to dispose of the used needle assembly after the drug is administered.

Storing and maintaining multiple containers in any medical environment requires space and may increase the time required to perform a procedure because the containers may become separated and must be located. Furthermore, storing and locating a third container for disposal of used needle assemblies requires additional space to accommodate the third container and may increase the time to perform a procedure by increasing the amount of time required to locate and access the third container.

### SUMMARY

In one aspect, a storage and dispensing container comprises a base having an open end and a receptacle. A first divider is secured in the receptacle, and a second divider is pivotally secured to the first divider. The first and second dividers define a first compartment having a size that is selectively adjustable by pivoting the second divider relative to the first divider.

In another aspect, an apparatus to facilitate separating a needle from its sheath comprises a body and a retaining structure positioned on the body configured to retain a sheath of a needle to facilitate separating the sheath from the needle. The retaining structure is configured to retain the sheath while the needle is in use and to facilitate covering the needle with the sheath after use of the needle.

Other aspects of the present invention will be apparent in view of the following description and claims.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a schematic perspective of a storage and dispensing container of a first embodiment of the present invention;

FIG. 2 is a top plan of the container of FIG. 1;

FIG. 3 is a top plan of a base of the container of FIGS. 1 and 2, in a first or empty configuration;

FIG. 4 is a top plan of the base of FIG. 3, in a second configuration;

FIG. 5 is a top plan of the base of FIGS. 3 and 4, including needle assemblies, cartridges and an expandable bag;

FIG. 6 is a top plan of a base of a storage and dispensing container according to a second embodiment of the present invention;

FIG. 7 is a section of the base of FIG. 6, taken along line 7-7;

FIG. 8 is an enlarged view of portion 8 of FIG. 7;

FIG. 9 is a perspective of a holding container according to an embodiment of the present invention, illustrating a syringe positioned therein;

FIG. 10 is an enlarged view of a distal portion of the syringe of FIG. 9;

FIG. 11 is a side elevation of a holding cushion according to an embodiment of the present invention, illustrating a syringe positioned therein;

FIG. 12 is a perspective of a first embodiment of a block for facilitating the uncapping and recapping of a needle assembly;

FIG. 13 is a perspective of a second embodiment of a block for facilitating uncapping and recapping of a needle assembly;

FIG. 14 is a section of the block of FIG. 13;

FIG. 15 is a side elevation of a third embodiment of a block for facilitating the uncapping and recapping of a needle assembly;

FIG. 16 is a side elevation of a fourth embodiment of a block for facilitating uncapping and recapping of a needle assembly; and

FIG. 17 is a side elevation of the block of FIG. 16.

Corresponding reference characters indicate corresponding parts throughout the drawings.

### DETAILED DESCRIPTION

The present invention is directed to a system and method for storing, dispensing and disposing of medical cartridges and disposable needles for use with reusable syringes. In one aspect of the invention, eliminates or reduces the number of containers necessary during a procedure by providing a container capable of storing and dispensing both drug cartridges and needle assemblies. Another embodiment may further reduce the number of containers used during a procedure by providing a storage and dispensing container which is also configured for the disposal of used needle assemblies. In addition to reducing the number of containers used during a single procedure which may increase a clinician's efficiency, another advantage of one embodiment of the present invention is the reduction of waste. Another advantage of one aspect of the present invention is the configuration of the container which may increase safety by reducing or eliminating the potential of accidental needle sticks upon recapping the used needle.

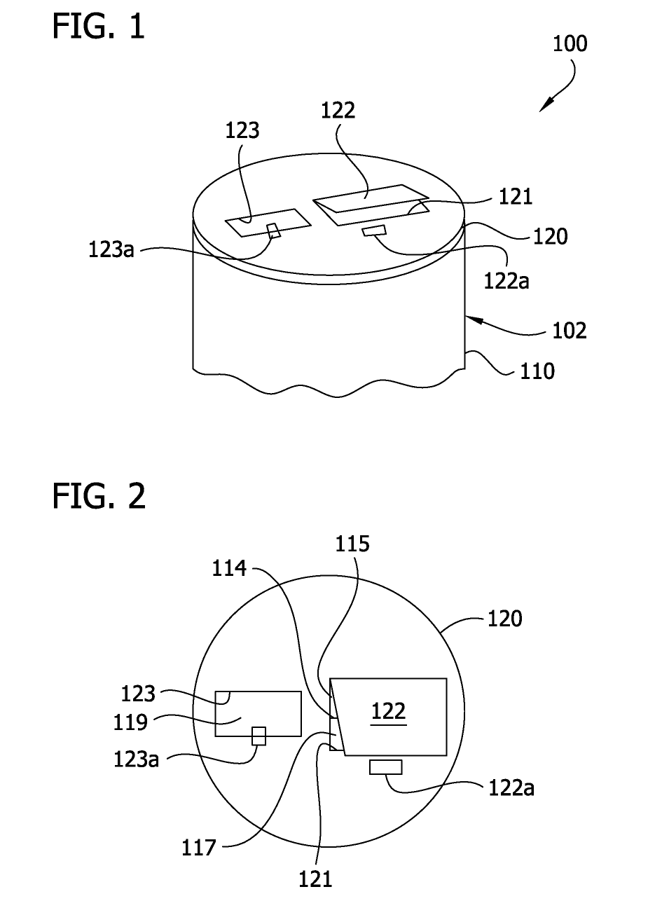

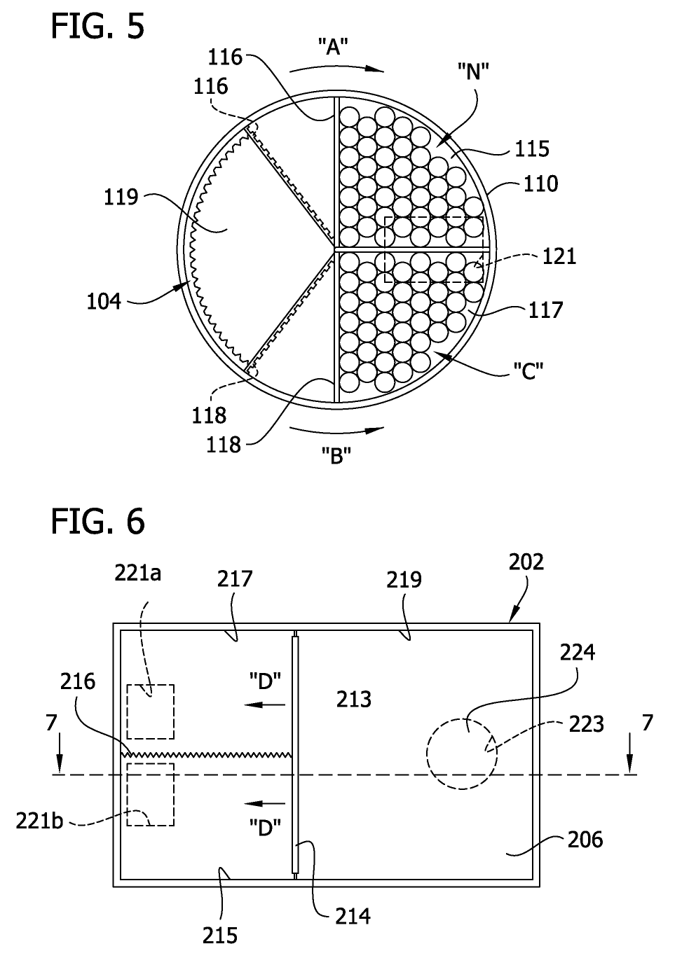

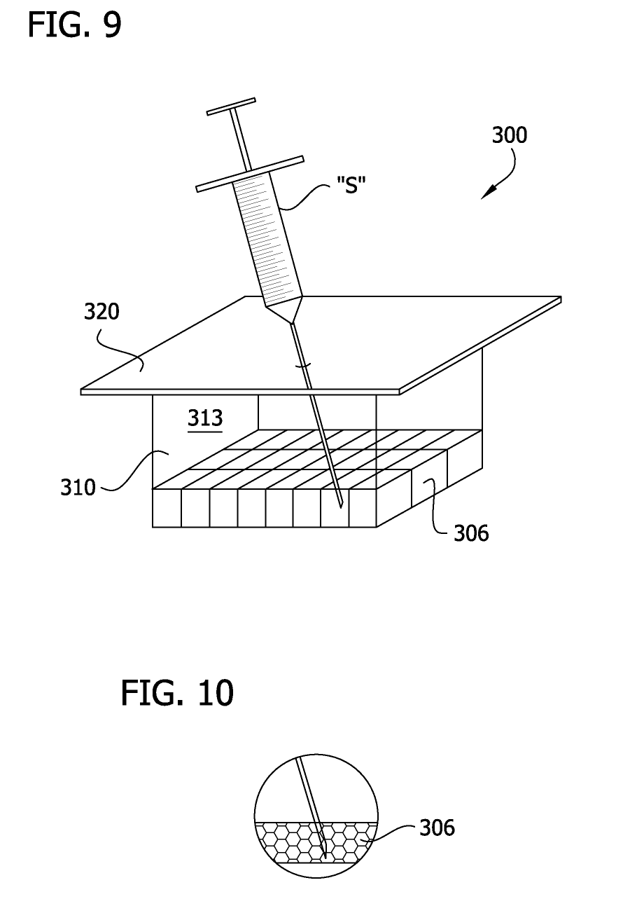

Referring to FIG. 1, one embodiment of a storage and dispensing system according to the present invention is designated in its entirety by the reference number 100. The container 100 is configured for storing and dispensing needle assemblies “N” (FIG. 5) and drug cartridges “C” (FIG. 5) for use with a reusable syringe “S” (FIG. 9). In one embodiment, drug cartridges “C” include local anesthetic cartridges (typically 1.8 mL glass cartridges filled with an anesthetic, e.g., lidocaine HCL 2% with epinephrine 1:100,000, available from suppliers such as Hospira, Septodont, Cook Waite) and needle assemblies “N” include dental needles (e.g., hard-pack needles available from Covidien). The container 100 is further configured for the disposal of the needle assemblies “N” after being used. The container 100 is typically provided to a user preloaded with a predetermined number, type, and size of needle assemblies “N” and cartridges “C”. It is envisioned, however, that the container 100 may be provided to the user empty and require loading. In this manner, the number, type, size and combination of needle assemblies “N” and cartridges “C” may be determined by the user. The container 100 may be disposable or reusable. Although the embodiments of the present invention are described as related to storage and dispensing needle assemblies and drug cartridges for use with reusable needles, it is envisioned that the containers of the present invention may be modified to store and dispense other medical components.

With reference to FIGS. 1-5, the container 100 includes a substantially cylindrical body 102 having a base 110 and a cover 120. Although illustrated as having a substantially cylindrical configuration, it is envisioned the container 100 may have other shapes, including, for example, cubic and ellipsoid. The base 110 and the cover 120 may be formed of plastic, polymer, metal, alloy, or other suitable material. The base 110 and/or the cover 120 may be translucent, transparent, or opaque. The base 110 and the cover 120 may be formed of the same or different materials. The cover 120 may be selectively secured to the base 120 by threads, mechanical fasteners, friction fit, or other suitable means. In addition, the cover 120 may be permanently affixed to the base 110 by bonding, welding, adhesives, or other suitable methods. The base 110 and/or the cover 120 may include a holder (not shown) for storing a reusable syringe (FIG. 9). In one embodiment, the holder is molded into the base 110 and/or the cover 120.

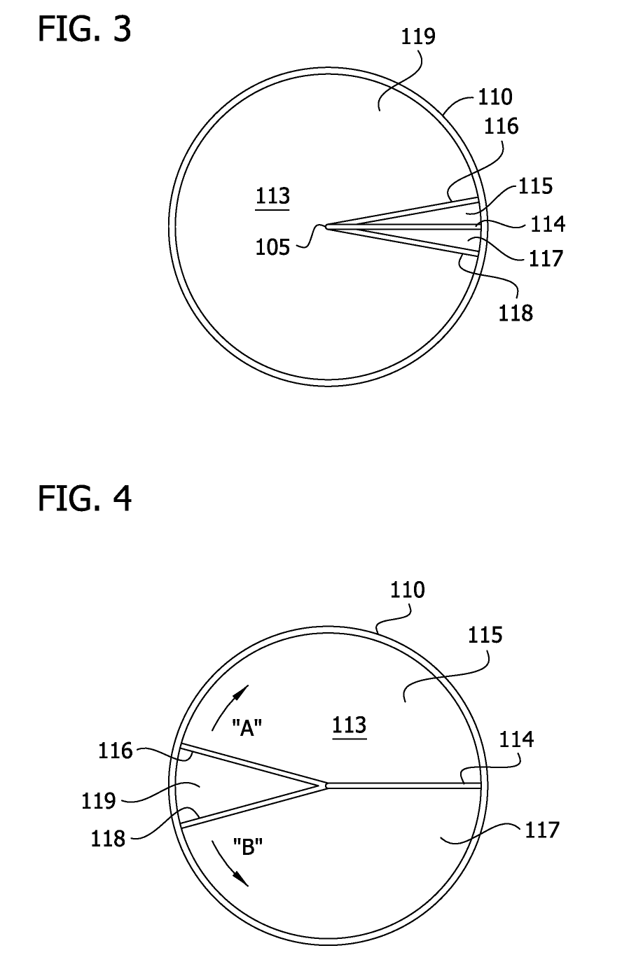

With particular reference to FIGS. 3 and 4, the base 110 defines a cavity or receptacle 113 including a plurality of dividers 114, 116, 118. The dividers 114, 116, 118 partition the cavity 113 into three compartments 115, 117, 119. The first compartment 115 is configured for storing and dispensing a plurality of needle assemblies “N” and may be selectively configured to retain any number of needle assemblies “N.” The second compartment 117 is configured for storing and dispensing a plurality of drug cartridges “C” and may be selectively configured to retain any number of cartridges “C.” The first and second compartments 115, 117 may be of equal or different sizes, and may also be sized to retain equal or different numbers of needle assemblies “N” and cartridges “C,” respectively. The third compartment 119 is configured for disposing used needle assemblies “N.”

The first divider 114 is rigidly secured in the cavity 113 of the base 110 and extends radially outward from a central pivot 105. The second and third dividers 116, 118 are pivotally secured to the first divider 114 at the central pivot 105 and extend radially outward from the central pivot 105 to the base 110. The second divider 116 is configured to pivot about the central pivot 105 and is biased clockwise, as indicated by arrow “A.” The third divider 118 is configured to pivot about the central pivot 105 and is biased counter-clockwise, as indicated by arrow “B.” The first compartment 115 is bounded by the first and second dividers 114, 116 and has a selectively adjustable size as the second divider 116 pivots relative to the first divider 114. The second compartment 117 is bounded by the first and third dividers 114, 118 and has a selectively adjustable size as the third divider 118 pivots relative to the first divider 114. The third compartment 119 is formed between the second and third dividers 116, 118 and is also of a selectively adjustable size as the second and third dividers 116, 118 pivot relative to each other.

Referring again to FIGS. 1 and 2, the cover 120 includes a first opening 121 positioned on the cover to facilitate access to at least a portion of the first and second compartments 115, 117 of the base 110 when the cover is secured to the base. The first opening 121 may include a lid 122 and optionally a lock 122a. Although the lock 122a is shown in an unlocked position as a pivoting arm that may be keyed, the lock may be any conventional internal or external lock. In this manner, the first opening 121 provides access to needle assemblies “N” (FIG. 5) and cartridges “C” (FIG. 5). In one embodiment, the lid 122 may be formed from two separate lids, each facilitating independent access to one of the first and second compartments 115, 117. In another embodiment, the first opening 121 may include separate openings for individually accessing the first and second compartments 115, 117.

The cover 120 may also include a second opening having a lid 123 and optionally a lock 123a configured for accessing the third compartment 117. Although the lock 123a is shown in a locked position as a pivoting arm which may be keyed, the lock may be a conventional internal or external lock. The cover 120 may include a separation mechanism (not shown) formed about the opening 123 to facilitate separating a used needle assembly “N” from a reusable syringe “S” (FIG. 9).

With reference now to FIG. 3, in an initial or empty configuration, the second and third dividers 116, 118 are pivoted about the central pivot 105 in clockwise and counter-clockwise directions, respectively, such that the second and third dividers are adjacent to the first divider 114. In the empty configuration, the first and second compartments 115, 117 are minimized while the third compartment 119 occupies the remainder of the base 110.

Referring now to FIG. 4, in a second configuration, the container 100 is configured for receiving needle assemblies “N” and cartridges “C” In the second configuration, the second and third dividers 116, 118 are pivoted in counter-clockwise and clockwise directions, respectively, such that the second and third dividers are nearest one another. In this manner, the first and second compartments 115, 117 are maximized to permit needle assemblies “N” and cartridges “C,” respectively, to be loaded. Loading the base 110 may occur before the cover 120 is secured to the base 110. Alternatively, needle assemblies “N” and cartridges “C” may be loaded into the base 110 through the first opening 121.

FIG. 5 illustrates the container 100 containing a plurality of needle assemblies “N” and a plurality of cartridges “C”. The third compartment 119 may include a bag 104 or other sealable body capable of preventing I liquid waste from the used needle assemblies “N” leaking of the compartment. The bag 104 may be expandable and/or stretchable such that the bag increases in size as the third compartment 119 expands and/or needle assemblies “N” are deposited. Alternatively, the base 110 and the cover 120 may be configured such that the third compartment 119 is sealed to prevent fluid leakage.

As discussed above, the container 100 may be provided to a user preloaded with needle assemblies “N” and cartridges “C,” or may require loading. Once loaded, the lid 122 on the cover 120 is opened to allow a needle assembly “N” to be removed from the first compartment 115 and a cartridge “C” to be removed from the second compartment 117 through the first opening 121. As discussed above and as shown in FIG. 5, the second divider 116 is urged in a direction “A” about the central pivot 105 and the third divider 118 is urged in a direction “B” about the central pivot 105. As such, removal of needle assembly “N” from the first compartment 115 causes the second divider 116 to pivot clockwise, as indicated by arrow “A”, to move the remainder of needle assemblies “N” toward the opening 121. Likewise, removing a cartridge “C” from the second compartment 117 causes the third divider 118 to pivot counter-clockwise, as indicated by arrow “B”, to move the remainder of needle assemblies “N” toward the opening 121. In one embodiment, the base 110 includes a sloped surface (not shown) in each of the first and second compartments 115, 117 adjacent the first divider 114. The sloped surfaces are configured to elevate a respective needle assembly “N” or cartridge “C” relative to the other needle assemblies “N” or cartridges “C” within the respective first and second compartments 115, 117 to facilitate removal of the respective needle assembly “N” or cartridge “C” from the container 100 through the first opening 121.

As discussed above, pivoting of the second divider 116 clockwise causes the first compartment 115 to become smaller and pivoting of the third divider 118 counter-clockwise direction causes the second compartment 117 to become smaller, thereby enlarging the third compartment 119. As shown in FIG. 5, the bag 104 in the third compartment 119 is configured to expand and/or stretch as the second and third dividers 116, 118 pivot relative to the central pivot 105. Once the drug has been administered, the needle assembly “N” may be recapped and disposed of in the third compartment 119 through the opening 123 in the cover 120. As discussed above, the cover 120 may be configured to facilitate separating the used needle assembly “N” from the reusable syringe “5”.

Referring to FIGS. 6-8, a second embodiment of a storage and dispensing system is shown generally as storage and dispensing container 200. The container 200 is substantially similar to the container 100 described above, and only differences between the container 200 and the container 100 will be described. The container 200 includes a substantially rectangular box 202 having a base 210 and a cover 220. The base 210 defines a cavity 213 and includes a rigid first divider 214 and a flexible second divider 216. The first divider 214 extends the width of the cavity 213 and is biased in a first direction, as indicated by arrows “D.” Alternatively, the first divider 214 may be fixed in place. The first divider 214 partitions the cavity 213, forming a first compartment 215, a second compartment 217, and a third compartment 219. The first divider 214 includes seal members 214a along its edges that form a fluid seal with the base 210. The seal members 214a (FIG. 8) function similarly to wiper blades, preventing fluid leakage from the third compartment 219 into the first and second compartments 215, 217.

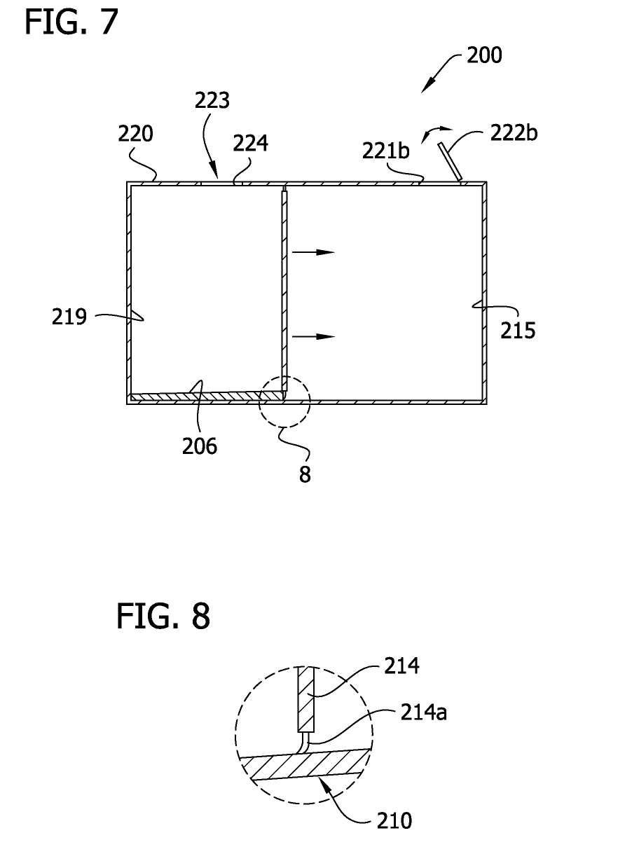

Still referring to FIGS. 6-8, the collapsible second divider 216 extends longitudinally between a wall of the base 210 and the first divider 214. The second divider 216 separates the first and second compartments 215, 217 of the cavity 213. The first compartment 215 is configured for storing and dispensing a plurality of needle assemblies “N,” and the second compartment 217 is configured for storing and dispensing a plurality of cartridges “C.” The second divider 216 is configured to collapse as the first divider 214 is biased toward the first and second compartments 215, 217. One or more surfaces forming the third compartment 219 may include a layer of hydrogel adhesive 206 (FIG. 7) to secure used needle assemblies “N” to the floor of the compartment 219 when deposited after use. Hydrogel adhesive 206 may also absorb any fluids that may be present on the needle assemblies “N.”

Although the container 200 in FIG. 6 is shown to be rectangular, the container may have any suitable shape including square, elliptical or circular. In one embodiment (not shown), the container is circular and comprises three concentric compartments separated by two concentric dividers. The first and the second dividers may be collapsible or expandable depending on the bias and the direction of movement of the divider. Each of the two dividers may be biased toward or away from the center of the container. Alternatively, one divider may be biased toward the center of the container and the other divider may be biased away from the center of the container.

With reference still to FIGS. 6-8, the cover 220 includes first and second openings 221a, 221b, respectively, for accessing the respective first and second compartments 215, 217. Either or both of the first and second openings 221a, 221b may include a lid 222b and optionally a lock (not shown). The cover 220 also includes a third opening 223 (FIG. 6) for accessing the third compartment 219. As shown, the third opening 223 includes a seal member 224 configured to permit a needle assembly “N” to be received into the third compartment 219 following use. In one embodiment, the seal member 224 may include a resilient material covering the opening 223 and a slit allowing a needle assembly “N” to pass. Alternatively, the seal member 224 may include a pivotal door and optionally a lock (not shown). The seal member 224 may further be configured to facilitate separating the used needle assembly “N” from the reusable syringe “S.”

Referring to FIGS. 9 and 10, an apparatus for holding a loaded syringe “S” includes a holding container 300. The holding container 300 includes a base 310 and a cover 320. The base 310 includes a substantially rigid body defining a cavity 313 and may be composed of plastic, polymer, or other suitable material and may be transparent, translucent, or opaque. The base 310 includes a gel matrix 306 configured for receiving the distal end of a needle of reusable syringe “S.” The gel matrix 306 may include a buffer solution, topical anesthetic, or other material. As seen in FIG. 10, the gel matrix 306 may have a honeycomb configuration.

The cover 320 of the holding container 300 is preferably formed of a pierceable material, such as Tyvek film. Tyvek is a U.S federally registered trademark of E.I. du Pont de Nemours and Company of Wilmington, Del., United States. The cover 320 is configured to receive a needle of a reusable syringe “S” and to maintain the syringe “S” in an upright position in anticipation of use. The cover 320 further operates to prevent any liquids from evaporating or leaking and/or to prevent contaminants from entering the holding container 300.

In use, the needle of a loaded syringe “S” is pierced through the cover 320 and lodged into the gel matrix 306. The cover 320 and the gel matrix 306 operate to maintain the syringe “S” in the upright position. When the syringe “S” is needed, the needle of the syringe “S” is withdrawn from the gel matrix 306 and through the cover 320. The holding container 300 may be used repeatedly during a procedure. It is envisioned that the holding container 300 may be incorporated into or used together with either or both of the storing and dispensing containers 100, 200 described above.

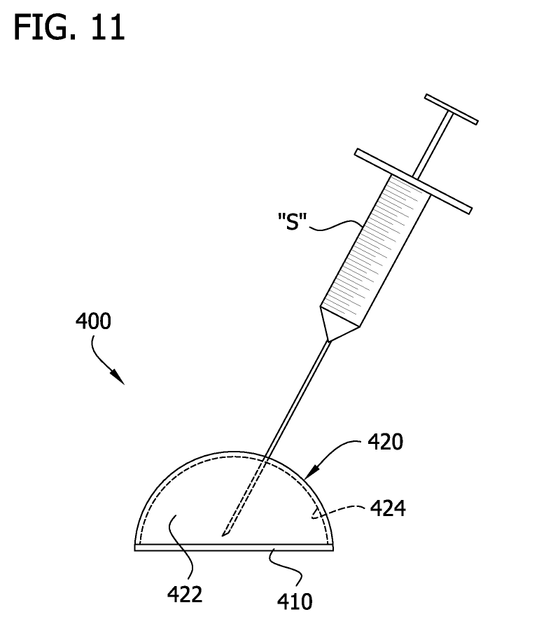

Referring now to FIG. 11, an alternate embodiment of an apparatus for holding a loaded syringe “S” is shown generally as holding cushion 400. The cushion 400 includes a base 410 and a cushion member 420. The base 410 is a substantially circular body configured to be selectively or securely attached to a working surface, i.e., an operating tray, or other location. The base 410 may include an adhesive pad, hook and loop fasteners, or other suitable fastening means to secure the base to the working surface. The cushion member 420 includes a foam block 422 and a pierceable cover 424. The foam block 422 may include a closed cell structure and may further include one or more materials for treating the needle of the syringe “S”, e.g., an anti-microbial or antiseptic. It is envisioned the holding cushion 400 may be incorporated into or used together with either or both of the storing and dispensing containers 100, 200 described above.

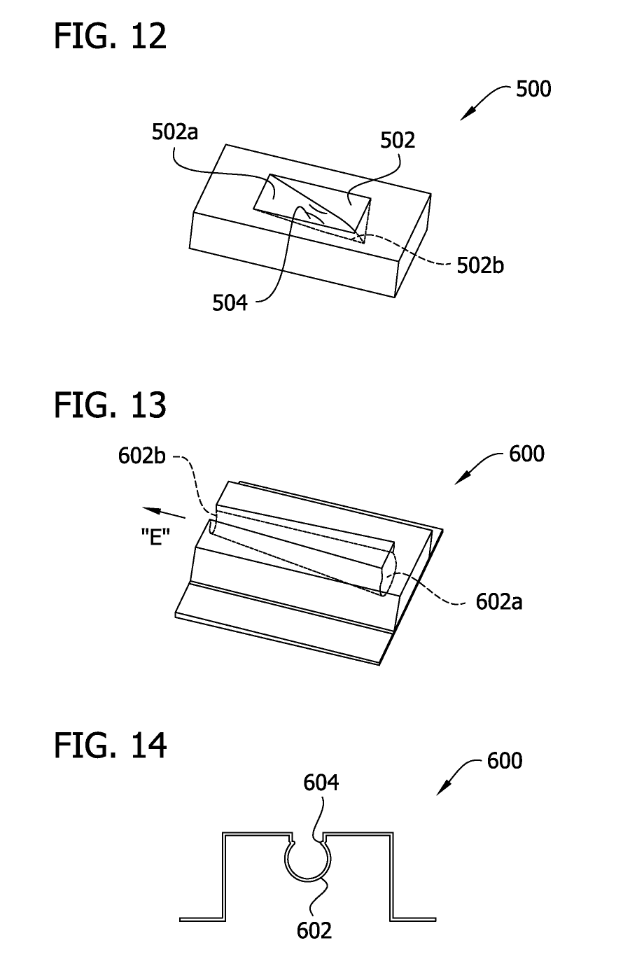

FIGS. 12-17 illustrate an apparatus to facilitate separating a hypodermic needle from its sheath, maintaining the sheath, and facilitating the re-capping of the needle following use. Referring initially to FIG. 12, a block 500 has a sloped plane 502 having shallow and deep ends 502a, 502b, respectively. The length of the sloped plane 502 is slightly shorter than the length of a respective needle sheath, such that the opening in the sheath will be elevated slightly higher than the block 500. The block 500 may be formed of plastic, polymer, or other suitable material. The block 500 further includes indents 504 formed along the sloped plane 502. The indents 504 are configured to frictionally engage a needle sheath to facilitate separating the sheath from the needle, to retain the sheath during use of the syringe “S” and to facilitate receiving the needle back into the sheath after use.

Referring to FIGS. 13 and 14, an alternate embodiment of an apparatus to facilitate separating, maintaining, and re-capping a needle assembly “N” is shown generally as block 600. The block 600 has an elongate cylindrical slot 602 that tapers from a first end 602a to a second end 602b. The elongate slot 602 is configured to receive a needle assembly “N” of a reusable syringe “S”. The block 600 includes a ridge 604 formed near the second end 602b of the elongate slot 602. The ridge 604 is configured to facilitate separating a sheath from a needle assembly “N”. In use, a needle assembly “N” having a sheath is loaded in the elongate slot 602. As the syringe “S” of the needle assembly “N” is pulled in the direction of the arrow “E”, the ridge 604 engages the sheath of the needle assembly, and separates the sheath from the syringe. The sheath remains in the elongate slot 602 while the syringe “S” is used. The syringe “S” may then be recapped by inserting the needle of the syringe through the second end 602b of the elongate slot 602 and into the sheath.

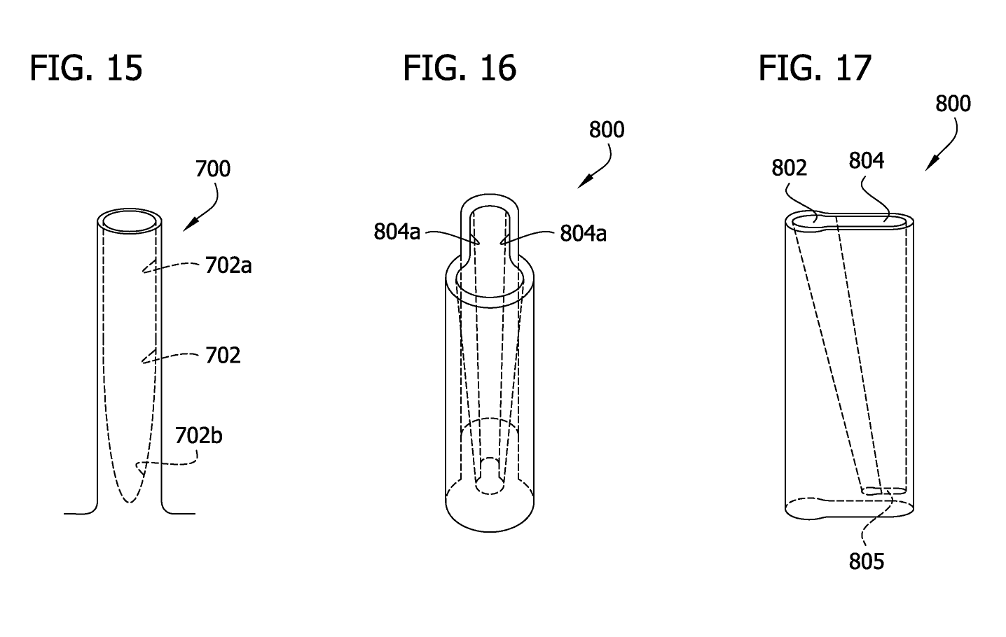

Referring now to FIG. 15, another embodiment of an apparatus for facilitating separating, maintaining, and re-capping a needle assembly “N” is shown generally as block 700. The block 700 has a recess 702 that tapers from a first end 702a to a second end 702b. The recess 702 is configured to receive a needle assembly “N” of a reusable syringe “S”. The tapered configuration of the recess 702 permits the block 700 to frictionally engage the sheath of the needle assembly “N.” The recess 702 is configured such that the frictional engagement of the sheath is sufficient to permit the sheath to be disengaged from the syringe “S”, thereby separating of the sheath from the syringe as the syringe is retracted from the block 700. The syringe “S” may be recapped in the reverse manner.

With reference now to FIGS. 16 and 17, yet another embodiment of an apparatus to facilitate separating, maintaining, and re-capping a needle assembly “N” is shown generally as block 800. The block 800 has first and second elongate tapered recesses 802, 804. The first recess 802 is configured to receive a needle assembly “N” including a sheath. The block 800 includes a lip 804a formed about a first end of the second recess 804. The lip 804a is configured to engage the sheath of the needle assembly “N” and to facilitate the sheath separating from the needle assembly. The second recess 804 may include an open second end 805 configured for accessing a compartment for disposing the needle assembly “N” after use.

Although the illustrative embodiments of the present disclosure have been described herein with reference to the accompanying drawings, it is to be understood that the disclosure is not limited to those precise embodiments, and that various other changes and modifications may be effected therein by one skilled in the art without departing from the scope or spirit of the disclosure. For example, any of the above described holding devices or blocks may be incorporated into any of the above described storage and dispensing containers.

The present invention eliminates or reduces the number of containers necessary during a procedure by providing a container capable of storing and dispensing both drug cartridges and needle assemblies. The present invention may further reduce the number of containers used during a procedure by providing a storage and dispensing container that is also configured for disposing of used needle assemblies. In addition to reducing the number of containers used during a single procedure, which may increase a user's efficiency, another advantage of the present invention is reducing waste. Further, the configuration of the container may increase safety by reducing or eliminating the potential for accidental needle sticks when recapping used needles.

Having described the invention in detail, it will be apparent that modifications and variations are possible without departing from the scope of the invention defined in the appended claims.

When introducing elements of the present invention or the preferred embodiments thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A storage and dispensing container comprising:
a base having an open end and a receptacle;
a first divider secured in the receptacle; and
a second divider pivotally secured to the first divider, wherein the first and second dividers define a first compartment, the first compartment having a size that is selectively adjustable by pivoting the second divider relative to the first divider.

2. The storage and dispensing container of claim 1, further comprising a third divider pivotally secured to the first divider, wherein the first and third dividers define a second compartment, the second compartment having a size that is selectively adjustable by pivoting the third divider relative to the first divider, and wherein the second and third dividers define a third compartment, the third compartment having a size that is selectively adjustable by pivoting the second and third dividers.

3. The storage and dispensing container of claim 2, wherein the first compartment is adapted to receive a plurality of needle assemblies, and wherein removal of a needle assembly from the first compartment causes the second divider to pivot relative to the first divider, thereby reducing the size of the first compartment and increasing the size of the third compartment.

4. The storage and dispensing container of claim 3, wherein the second compartment is adapted to receive a plurality of cartridges filled with medicament, and wherein removal of a cartridge from the second compartment causes the third divider to pivot relative to the first divider, thereby reducing the size of the second compartment and increasing the size of the third compartment.

5. The storage and dispensing container of claim 1, further comprising a cover configured to close the open end of the base, the cover having a first opening configured to permit access to the first compartment.

6. The storage and dispensing container of claim 2, further comprising a cover configured to close the open end of the base, the cover having a first opening configured to permit access to the first and second compartments and a second opening configured to permit access to the third compartment.

7. The storage and dispensing container of claim 1, wherein the second divider is biased toward the first divider.

8. The storage and dispensing container of claim 2, wherein the second divider is biased in a first direction toward the first divider, and the third divider is biased in a second direction opposite the first direction toward the first divider.

9. The storage and dispensing container of claim 2, further comprising a bag positioned in the third compartment and configured to receive needle assemblies after use, the bag being expandable to fill the third compartment as the size of the third compartment is increased.

10. The storage and dispensing container of claim 5, wherein at least one of the base and the cover includes a holder for retaining a loaded syringe.

11. The storage and dispensing container of claim 10, wherein the base includes a gel matrix configured to receive a distal end of a needle to hold a loaded syringe.

12. The storage and dispensing container of claim 11, wherein the needle is pierced through the cover and into the gel matrix to retain the loaded syringe in an upright position.

13. An apparatus to facilitate separating a needle from its sheath, the apparatus comprising:
a body; and
a retaining structure positioned on the body configured to retain a sheath of a needle to facilitate separating the sheath from the needle, wherein the retaining structure is configured to retain the sheath while the needle is in use and to facilitate covering the needle with the sheath after use of the needle.

14. The apparatus of claim 13, further comprising a sloped plane on the body, wherein the retaining structure comprises an indent formed on the sloped plane configured to frictionally engage the sheath.

15. The apparatus of claim 13, further comprising an elongate slot on the body, the elongate slot being cylindrical and tapering from a first end to a second end.

16. The apparatus of claim 15, wherein the retaining structure comprises a ridge formed near the second end of the elongate slot.

17. The apparatus of claim 13, wherein the retaining structure comprises a tapered recess in the body.

18. The apparatus of claim 13, wherein the body has first and second tapered recesses.

19. The apparatus of claim 18, wherein the retaining structure comprises a lip formed about a first open end of the second tapered recess.

20. The apparatus of claim 19, wherein the second recess includes a second open end opposite the first open end configured to permit access to a compartment for disposing a needle after use.
